(19) United States
(12) Patent Application Publication
(10) Pub. No.: US 2026/0102550 A1
(43) Pub. Date: Apr. 16, 2026

(54) METHOD AND SYSTEM FOR DETECTING FLUID LEVEL IN VESSEL CONNECTED TO PATIENT

(71) Applicant: Cloudphysician Healthcare Pvt. Ltd., Bangalore (IN)

(72) Inventors: T. Senthur Ram, Namakkal (IN); Jitesh Sekar, Bengaluru (IN); Pradip Maganbhai Solanki, Rajkot (IN); Dhruv Sud, Bengaluru (IN)

(21) Appl. No.: 19/356,250

(22) Filed: Oct. 13, 2025

(30) Foreign Application Priority Data
Oct. 11, 2024 (IN) 202441077370

(51) Int. Cl. A61M 1/00

(52) U.S. Cl. CPC A61M 1/69; A61M 2205/18; A61M 2205/3306; A61M 2205/3382; A61M 2205/3386; A61M 2205/3389; A61M 2205/6072

(57) ABSTRACT

This disclosure relates to method and system for detecting a fluid level in a fluid vessel connected to a patient. The method includes receiving a multimedia content associated with a patient, via at least one camera. The method further includes processing the multimedia content to detect a unique identifier associated with a bed of each patient. The method further includes analyzing the multimedia content to detect a fluid vessel attached to the bed of each patient. The method further includes estimating the fluid level within the fluid vessel connected to each patient. The method further includes iteratively updating a database based on the estimation and the unique identifier. The method further includes identifying an issue associated with the patients in response to the updating. The method further includes generating in real-time an alert to notify at least one person about the issue associated with the at least one patient.

### TECHNICAL FIELD

This disclosure generally relates to healthcare devices, and more particularly to a method and a system for detecting a fluid level in a fluid vessel connected to a patient.

### BACKGROUND

Globally, the healthcare sector is increasingly shifting towards modernization to provide advanced facilities, efficient management, and better decision-making. Urine bags are widely used in the medical sector for patients dealing with various health issues for example, kidney failure, bedridden (who are unable to take care of themselves), urinary incontinence, urinary retention, spiral cord injury, surgery, or blood clots in urine, monitoring overall fluid status in ICU patients, as a surrogate for monitoring kidney perfusion and oxygenation. Rapidly declining urine output is a direct indicator of critical diseases such as acute kidney injury which may necessitate either medications or interventions such as dialysis on an emergency basis. Detection of urine level in the urine bags is helpful for tracking a current health status of a patient and providing the precise treatment according to the current condition of the patient.

Currently, urine output detection and measurement heavily relies on manual observation by medical professionals, which are time consuming, may lead to human error, inaccuracy, carelessness, and the like. To counter these challenges, sensor-based urine bags like Wi-Uro have been developed, utilizing technologies such as Wi-Fi connectivity and integrated sensors. These existing sensor-based urine bags are able to provide a real-time monitoring of the urine bags, eliminate the need of regular manual observation, efficient, high efficiency, and time saving.

However, existing sensor-based urine bags face some challenges, such as consuming a lot of time for monitoring, easily affected by human error, complex design (e.g. integrating prototype sensor into urine bags involves additional complexity), expensive, and dependent on Wi-Fi connectivity.

Therefore, there is a need for implementing an efficient and reliable technique for detecting urine level in urine bags to address shortcomings of existing approaches employed in the medical sector.

### SUMMARY

In one embodiment, a method for detecting a fluid level in a fluid vessel connected to a patient is disclosed. In one example, the method may include receiving in real time, via at least one camera, a multimedia content associated with a set of patients within a room. The multimedia content may include at least one of an image or a video feed. The method may include processing the multimedia content to detect a unique identifier associated with a bed of each patient of the set of patients. The method may include analyzing, via a first Artificial Intelligence (AI) model, the multimedia content to detect a fluid vessel attached to the bed of each patient using a pre-defined detection technique. The method may include estimating, via the first AI model or an image processing algorithm, the fluid level within the fluid vessel connected to each patient using a pre-defined estimation technique. The method may include iteratively updating, via the first AI model, a database based on the fluid level estimated within the fluid vessel connected to each patient and the unique identifier, at a pre-defined time frame. The method may include identifying in real time an issue associated with the fluid level within the fluid vessel connected to at least one patient of the set of patients in response to the updating. The method may include generating in real time an alert to notify at least one person about the issue associated with the fluid level within the fluid vessel connected to at least one patient.

In another embodiment, a system for detecting a fluid level in a fluid vessel connected to a patient is disclosed. In one example, the system includes a processor and a computer-readable medium communicatively coupled to the processor. The computer-readable medium stores processor-executable instructions, which, on execution, may cause the processor to receive in real time, via at least one camera, a multimedia content associated with a set of patients within a room. The multimedia content may include at least one of an image or a video feed. The processor-executable instructions, on execution, may further cause the processor to process the multimedia content to detect a unique identifier associated with a bed of each patient of the set of patients. The processor-executable instructions, on execution, may further cause the processor to analyze, via a first Artificial Intelligence (AI) model, the multimedia content to detect a fluid vessel attached to the bed of each patient using a pre-defined detection technique. The processor-executable instructions, on execution, may further cause the processor to estimate, via the first AI model or an image processing algorithm, the fluid level within the fluid vessel connected to a patient using a pre-defined estimation technique. The processor-executable instructions, on execution, may further cause the processor to iteratively update, via the first AI model, a database based on the fluid level estimated within the fluid vessel connected to each patient and the unique identifier, at a pre-defined time frame. The processor-executable instructions, on execution, may further cause the processor to identify in real time an issue associated with the fluid level within the fluid vessel connected to at least one patient of the set of patients in response to the updating. The processor-executable instructions, on execution, may further cause the processor to generate in real time an alert to notify at least one person about the issue associated with the fluid level within the fluid vessel connected to at least one patient.

In yet another embodiment, a non-transitory computer-readable medium storing computer-executable instruction for detecting a fluid level in a fluid vessel connected to a patient is disclosed. The stored instructions, when executed by a processor, may cause the processor to perform operations including receiving, via at least one camera, multimedia content associated with a set of patients within a room. The multimedia content may include at least one of an images or a video feed. The operations may further include processing the multimedia content to detect a unique identifier associated with a bed of each patient of the set of patients. The operations may further include analyzing, via a first Artificial Intelligence (AI) model, the multimedia content to detect a fluid vessel attached to the bed of each patient using a pre-defined detection technique. The operations may further include estimating, via the first AI model or an image processing algorithm, the fluid level within the fluid vessel connected to each patient using a pre-defined estimation technique. The operations may further include iteratively updating, via the first AI model, a database based on the fluid level estimated within the fluid vessel connected to each patient and the unique identifier, at a pre-defined time frame. The operations may further include identifying in real-time an issue associated with the fluid level within the fluid vessel connected to at least one patient of the set of patients in response to the updating. The operations may further include generating in real-time an alert to notify at least one person about the issue associated with the fluid level within the fluid vessel connected to at least one patient.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this disclosure, illustrate exemplary embodiments and, together with the description, serve to explain the disclosed principles.

FIG. 1 is a block diagram of an exemplary system for detecting a fluid level in a fluid vessel connected to a patient, in accordance with some embodiments of the present disclosure.

FIG. 2 illustrates a functional block diagram of various modules within a memory of a detecting device configured to detect a fluid level in a fluid vessel connected to a patient, in accordance with some embodiments of the present disclosure.

FIG. 3 is a flow diagram of a process for detecting a fluid level in a fluid vessel connected to a patient, in accordance with some embodiments of the present disclosure.

FIG. 4 is a flow diagram of a process for creating a mapping dataset of a patient, in accordance with some embodiments of the present disclosure.

FIG. 5 illustrates an exemplary scenario for detecting a fluid level in a fluid vessel connected to a patient, in accordance with some embodiments of the present disclosure.

FIG. 6 is a block diagram of an exemplary computer system for implementing embodiments consistent with the present disclosure.

### DETAILED DESCRIPTION

Exemplary embodiments are described with reference to the accompanying drawings. Wherever convenient, the same reference numbers are used throughout the drawings to refer to the same or like parts. While examples and features of disclosed principles are described herein, modifications, adaptations, and other implementations are possible without departing from the spirit and scope of the disclosed embodiments. It is intended that the following detailed description be considered as exemplary only, with the true scope and spirit being indicated by the following claims.

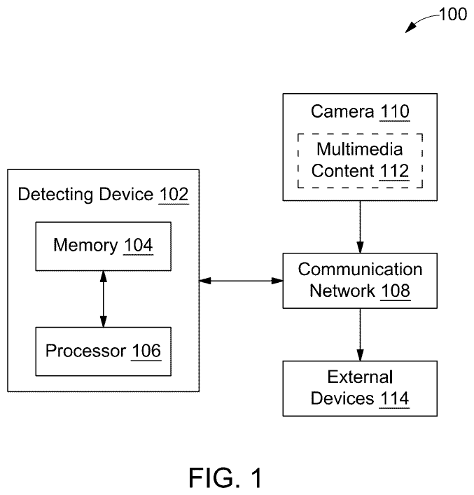

Referring now to FIG. 1, an exemplary system 100 for detecting a fluid level in a fluid vessel connected to a patient is illustrated, in accordance with some embodiments of the present disclosure. As depicted via the present FIG. 1, the system 100 may include a detecting device 102. The detecting device 102 may be configured to detect the fluid level in the fluid vessel connected to the patient. The detecting device 102 may be, for example, but may not be limited to, server, desktop, laptop, notebook, netbook, tablet, smartphone, mobile phone, or any other computing device), in accordance with some embodiments. In particular, a purpose of the detecting device 102 may be to accurately monitor and track the fluid level within the fluid vessel to ensure timely intervention and appropriate medical management.

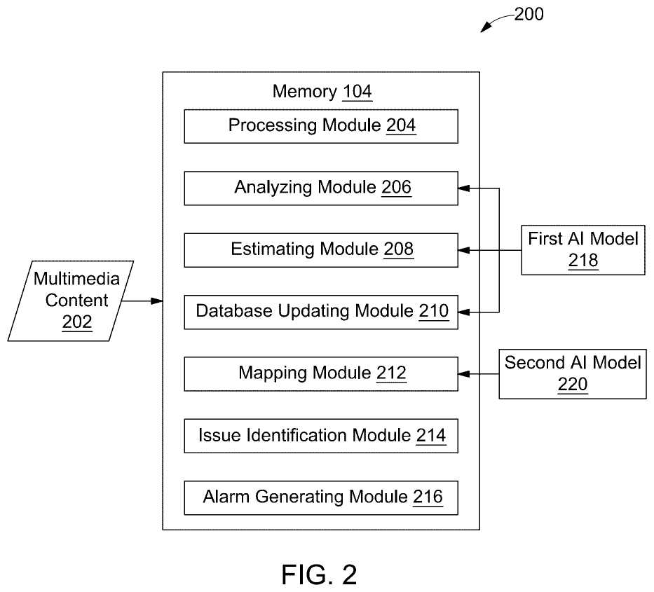

As will be described in greater detail in conjunction with FIG. 2-4, in order to detect the fluid level in the fluid vessel connected to the patient, the detecting device 102 may initially receive in real time a multimedia content 112 associated with a set of patients within a room, via at least one camera 110. The multimedia content may include at least one of an images or a video feed. The detecting device 102 may further process the multimedia content 112 to detect a unique identifier associated with a bed of each patient of the set of patients. Detection device 102 may further analyze the multimedia content 112 to detect a fluid vessel attached to the bed of each patient through a first AI model using a pre-defined detection technique. Detecting device 102 may further estimate the fluid level within the fluid vessel connected to each patient through the first AI model or an image processing algorithm using a pre-defined estimation technique. The detecting device 102 may further iteratively update, via the first AI model, a database based on the fluid level estimated within the fluid vessel connected to each patient and the unique identifier, at a pre-defined time frame. The detecting device 102 may further identify in real-time an issue associated with the fluid level within the fluid vessel connected to at least one patient of the set of patients in response to the updating. The detecting device 102 may further generate in real-time an alert to notify at least one person about the issue associated with the fluid level within the fluid vessel connected to at least one patient.

In some embodiments, the detecting device 102 may include one or more processors 106 and a memory 104. Memory 104 may store instructions that, when executed by the one or more processors 106, cause the one or more processor 106 to detect the fluid level in the fluid vessel connected to the patient, in accordance with aspects of the present disclosure. The memory 104 may further store various data (for example, video data, image data, fluid related data associated with the patients, training data, historical medical data, medical record number (MRN), and personal details of the patient (e.g., name, address, allotted bed number, name of diseases, blood group, weight, height, etc.)) that may be captured, processed, and/or required by the system 100. The memory 104 may be non-volatile memory (e.g., flash memory, Read Only Memory (ROM), Programmable ROM (PROM), Erasable PROM (EPROM), Electrically EPROM (EEPROM) memory, etc.) or a volatile memory (e.g., Dynamic Random Access Memory (DRAM), Static Random-Access memory (SRAM, etc.). Memory 104 may also include a database (not shown in FIG. 1) for storing data.

System 100 may further include at least one camera 110 to capture a real time multimedia content 112 (e.g., image or video feed) associated with a set of patients. System 100 may further include a communication network 108. In some embodiments, the detecting device 102 may receive multimedia content 112 via at least one camera 110 over the communication network 108.

The system 100 may further include one or more external devices 114. In some embodiments, the detecting device 102 may send various data related to the patient to the one or more external devices 114 over the communication network 108. The communication network 108 may include, for example, but not limited to, a wireless fidelity (Wi-Fi) network, a light fidelity (Li-Fi) network, a local area network (LAN), a wide area network (WAN), a metropolitan area network (MAN), a satellite network, the Internet, a fiber optic network, a coaxial cable network, an infrared (IR) network, a radio frequency (RF) network, and a combination thereof. The one or more external devices 114 may include, for example, but may not be limited to, server, laptop, netbook, notebook, smartphone, mobile phone, tablet, or any other computing device.

Referring now to FIG. 2, a functional block diagram 200 of various modules within memory 104 of the detecting device 102 is illustrated, in accordance with some embodiments of the present disclosure. FIG. 2 is explained in conjunction with FIG. 1. To detect the fluid level in the fluid vessel connected to the patients, the memory 104 may include a processing module 204, an analyzing module 206, an estimating module 208, a database updating module 210, a mapping module 212, an issue identification module 214, and an alarm generating module 216.

In order to detect the fluid level in the fluid vessel connected to the patients, the processing module 204 may initially receive in real time a multimedia content 202 associated with a set of patients within a room. The multimedia content may be at least one of an images or a video feed captured via at least one camera 110 present within the room.

The set of patients may include, for example, but may not be limited to, one or more patients with chronic conditions such as heart failure, kidney disease, or sepsis that may require ongoing monitoring of fluid status to guide treatment decisions and optimize outcomes. The rooms may include hospital rooms, for example, but may not be limited to, Intensive Care Unit (ICU), general wards, emergency wards, private wards, operation theatre (OT), or home premises rooms. At least one camera 110 may automatically adjust to a specific position to accurately capture the image or video feed of the patients lying on a bed of the room. By way of an example, one or more cameras may be installed in the ICU to capture a real time image or video feed associated with the patients.

Upon receiving in real time, multimedia content 202 associated with the set of patients, the processing module 204 may process the multimedia content 202 to detect a unique identifier associated with the bed of each patient of the set of patients. The unique identifier may include, for example, but may not be limited to, a Quick Response (QR) code, a bar code, a Radio Frequency Identification (RFID) tag, a unique bed number, simultaneous localization and mapping (SLAM).

In some embodiments, to process the multimedia content 202, the processing module 204 may be configured to extract information associated with each patient from the multimedia content 202 based on the unique identifier. The information may include, but not limited to, the bed number of each patient, and medical record number (MRN) of each patient. Additionally, in some embodiments upon accessing the MRN, personal details of each patient may be obtained. The personal details may include, for example, but not limited to, name of the patient, address of the patient, age of the patient, gender of the patient, blood group of the patient, weight of the patient, and height of the patient.

The analyzing module 206 may employ a first AI model 218 to analyze the multimedia content 202 to detect a fluid vessel attached to the bed of each patient using a pre-defined detection technique. The pre-defined detection technique may include, for example, but not limited to, a Convolution Neural Network (CNN), a Region Based Convolutional Neural Network (R-CNN), a Residual Network (ResNet), a Single Shot Multibox Detector (SSD), and a You Only Look Once (YOLO). In some embodiments, the fluid vessel may include, for example, but not limited to, catheter leg bags, drainage bags, texas catheter bags, night drainage bags, and saline bags.

Further, the estimating module 208 may employ the first AI model 218 (or an image processing algorithm) to estimate a fluid level within the fluid vessel connected to each patient using a pre-defined estimation technique. The pre-defined estimation technique may include, for example, but not limited to, a computer vision technique, and a machine learning technique. In some embodiments, the fluid level within the fluid vessel may include, but not limited to, urine level, glucose level, blood level, and the like.

Further, the database updating module 210 may employ the first AI model 206 to iteratively update a database based on the fluid level estimated within the fluid vessel connected to each patient and the unique identifier, in a pre-defined time frame. The database may include data related to, but not limited to, fluid level, MRN of the patients, personal details of the patients, bed number of the patients, and information about the fluid vessel.

In some embodiments, the mapping module 212 may employ a second AI model 220 to map the bed number to the MRN of each patient to create a mapping dataset. The MRN of each patient may include personal details, for example, but not limited to, name of the patient, address of the patient, age of the patient, gender of the patient, weight of the patient, height, and blood group of the patient. The mapping module 212 may further store the mapping dataset associated with each patient of the set of patients within the database.

The issue identification module 214 may be configured to identify in real time an issue associated with the fluid level within the fluid vessel connected to at least one patient of the set of patients in response to the updating. The issue may include various scenarios, for example, when the fluid level exceeds a pre-defined fluid level within the fluid vessel, indicating primary diseases which leads to a high fluid output state, a need for optimization of fluid administration or titration of medications like diuretics which increase one's fluid excretion. Conversely, when the fluid level falls below the pre-defined fluid level within the fluid vessel, suggesting dehydration, malfunctioning kidneys, hypovolemia, or inadequate fluid intake, which may necessitate quick intervention to prevent hemodynamic instability or organ dysfunction. In cases, when there is no detection of fluid level within the vessel, indicating equipment malfunction, disconnection, or other technical issues requiring immediate attention to ensure accurate monitoring and patient safety.

Upon identifying the issue, the alarm generating module 216 may generate in real time an alert to notify at least one person about the issue associated with the fluid level within the fluid vessel connected to at least one patient. The person may be, for example, but not limited to, a doctor, a nurse, a ward boy, and a caretaker. The alert may include, for example, but not limited to, an alarm, a text message, an email, and a phone call.

In some embodiments, the generation of alert may be customized based on individual characteristics and medical history of each patient. For instance, if a patient is dialysis dependent and thus does not produce urine, no alert may be generated, as this absence of urine output is expected and does not indicate a deviation from the norm. Conversely, for patients who are not dialysis dependent, alerts may be generated in the event of either insufficient or excessive urine output, as these deviations may signal issues requiring clinical attention. Therefore, the alarm generating module 216 ensures that alerts may be selectively generated for patients who exhibit variations in urine output that are clinically relevant, thereby optimizing the efficiency and effectiveness of the system.

It should be noted that all such aforementioned modules 202-216 may be represented as a single module or a combination of different modules. Further, as will be appreciated by those skilled in the art, each of the modules 202-216 may reside, in whole or in parts, on one device or multiple devices in communication with each other. In some embodiments, each of the modules 202-216 may be implemented as a dedicated hardware circuit comprising custom application-specific integrated circuit (ASIC) or gate arrays, off-the-shelf semiconductors such as logic chips, transistors, or other discrete components. Each of the modules 202-216 may also be implemented in a programmable hardware device such as a field programmable gate array (FPGA), programmable array logic, programmable logic device, and so forth. Alternatively, each of the modules 202-216 may be implemented in software for execution by various types of processors (e.g., processor 106). An identified module of executable code may, for instance, include one or more physical or logical blocks of computer instructions, which may, for instance, be organized as an object, procedure, function, or other construct. Nevertheless, the executables of an identified module or component need not be physically located together but may include disparate instructions stored in different locations which, when joined logically together, include the module, and achieve the stated purpose of the module. Indeed, a module of executable code could be a single instruction, or many instructions, and may even be distributed over several different code segments, among different applications, and across several memory devices.

As will be appreciated by one skilled in the art, a variety of processes may be employed for detecting a fluid level in a fluid vessel connected to a patient. For example, the exemplary system 100 and the associated detecting device 102, may detect the fluid level in the fluid vessel connected to the patient by the processes discussed herein. In particular, as will be appreciated by those of ordinary skill in the art, control logic and/or automated routines for performing the techniques and steps described herein may be implemented by the system 100 and the associated detecting device 102, either by hardware, software, or combinations of hardware and software. For example, suitable code may be accessed and executed by the one or more processors on the system 100 to perform some or all of the techniques described herein. Similarly, application specific integrated circuits (ASICs) configured to perform some, or all of the processes described herein may be included in the one or more processors on the system 100.

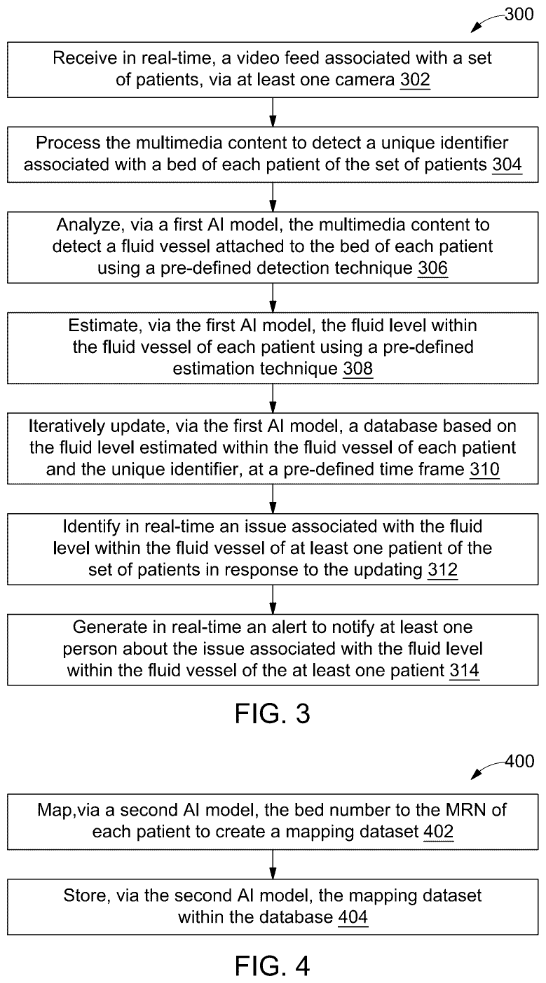

Referring now to FIG. 3, a process 300 for detecting a fluid level in a fluid vessel connected to a patient is illustrated via a flow chart, in accordance with some embodiments of the present disclosure. The process 300 may be implemented by the detecting device 102 of the system 100. The process 300 includes receiving in real time, a multimedia content 112 associated with a set of patients within a room, via at least one camera 110, at step 302. The multimedia content may include at least one of an image or a video feed. The set of patients may include, for example, but may not be limited to, one or more patients with chronic conditions such as heart failure, kidney disease, or sepsis that may require ongoing monitoring of fluid status to guide treatment decisions and optimize outcomes. The rooms may include hospital rooms, for example, but may not be limited to, Intensive Care Unit (ICU), general wards, emergency wards, private wards, operation theatre (OT), or home premises rooms.

Further, the process 300 includes processing the multimedia content 112 to detect a unique identifier associated with a bed of each patient of the set of patients, at step 304. The unique identifier may include a Quick Response (QR) code, a bar code, a Radio-Frequency Identification (RFID) tag, a unique bed number, and a simultaneous localization and mapping (SLAM). In some embodiments, for processing the multimedia content 112, process 300 may include extracting information associated with each patient from the multimedia content 112 based on the unique identifier. The information may include, for example, but not limited to, a bed number, and MRN associated with each patient.

Further, process 300 includes analyzing the multimedia content 112 to detect a fluid vessel attached to the bed of each patient through a first AI model 218 using a pre-defined detection technique, at step 306. The pre-defined detection technique may include, for example, but not limited to, a Convolution Neural Network (CNN), a Region Based Convolutional Neural Network (R-CNN), a Residual Network (ResNet), a Single Shot Multibox Detector (SSD), and a You Only Look Once (YOLO).

Further, the process 300 includes estimating the fluid level within the fluid vessel connected to each patient through the first AI model 218 (or an image processing algorithm) using a pre-defined estimation technique, at step 308. The pre-defined estimation technique may include, for example, but not limited to, a computer vision technique, and a machine learning technique.

Further, process 300 includes iteratively updating a database based on the fluid level estimated within the fluid vessel connected to each patient and unique identifier through the first AI model at a pre-defined time frame, at step 310.

Further, process 300 includes identifying in real time an issue associated with the fluid level within the fluid vessel connected to at least one patient of the set of patients in response to updating, at step 312.

Further, process 300 includes generating in real time an alert to notify at least one person about the issue associated with the fluid level within the fluid vessel connected to the at least one patient, at step 314. The alert may be generated when the fluid level estimated within the fluid vessel connected to each of the at least one patient for a timeframe is above or below a pre-defined fluid level. The person may include, for example, a doctor, a nurse, a ward boy, and the like. The alert may be generated in the form of alarm, message, email, and the like. In some embodiments, the alert generation may be customized based on the disease condition of each of the at least one patient.

Referring now to FIG. 4, a process 400 for creating a mapping dataset associated with the details of a patient is illustrated via a flow chart, in accordance with some embodiments of the present disclosure. As mentioned in FIG. 3, in response to step 304 of process 300, after processing the multimedia content 112 to detect a unique identifier associated with a bed of each patient of the set of patients, the process 400 includes mapping the bed number to the MRN of each patient to create a mapping dataset through the second AI model 220, at step 402.

The mapping dataset may include details of each patient mapped with allotted bed number. By way of an example, bed number-1 is mapped with the MRN of patient (XYZ) and bed number-2 is mapped with the MRN of patient (ABC). Further, process 400 includes storing the mapping dataset within the database through the second AI model, at step 404.

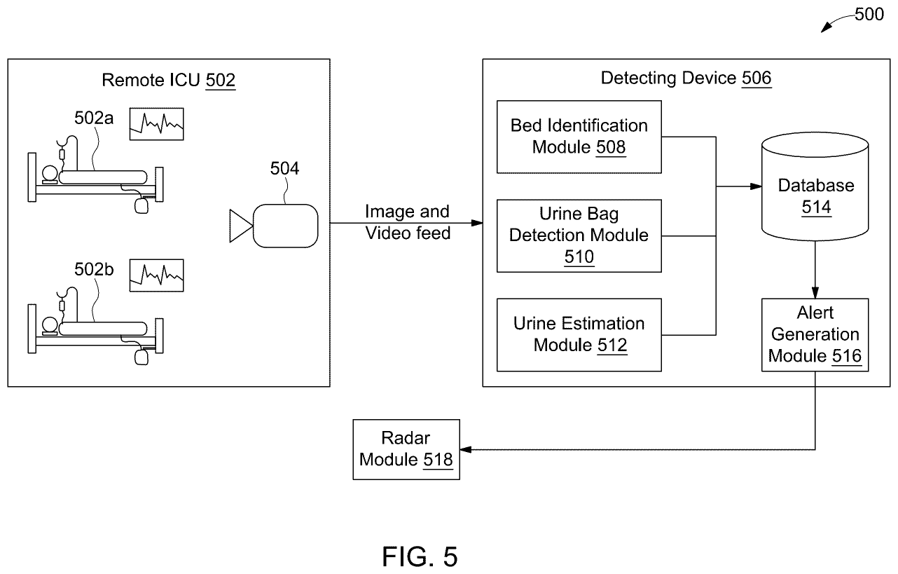

Referring now to FIG. 5, an exemplary scenario 500 for detecting a fluid level in a fluid vessel connected to a patient is illustrated via a block diagram, in accordance with some embodiments of the present disclosure. In the exemplary scenario 500, a set of patients (e.g., a first patient 502a and a second patient 502b) are admitted in an ICU 502.

A camera 504 may be installed in the ICU 502 that may capture real-time multimedia content (i.e., image and video feed) associated with the first patient 502a and the second patient 502b. Further, the image and video feed may be received by a detecting device 506 (analogous to the detecting device 102) to detect the fluid level in the fluid vessel connected to the first patient 502a and the second patient 502b.

In order to detect the fluid level, initially a bed identification module 508 may identify a bed number associated with each bed of patients. For example, the bed identification module 508 may identify that the patient 502a has been allotted a bed number 1, and the patient 502b has been allotted a bed number-2. The bed may be identified by either detecting a unique identifier (e.g., QR code) associated with the bed of each patient of the set of patients, or by combining images to understand ICU environment and map bed number to MRN of each patient.

Further, a urine bag detection module 510 may detect a urine bag attached to the bed of each patient. The urine bag may be detected using one of a pre-defined detection technique, for example, a CNN, a R-CNN, a ResNet, an SSD, and a YOLO. For example, the pre-defined detection technique may detect that patient 502a and the patient 502b have urine bags attached to their beds respectively.

Further, a urine estimating module 512 may estimate the urine level within the urine bag of each patient. The urine level may be estimated using one of a pre-defined estimation technique, for example, computer vision and a machine learning. In continuation with the above example, the pre-defined estimation technique may estimate that the patient 502a has urine level (e.g. 1500 ml) in urine bag attached to the bed number 1, and patient 502b has urine level (e.g. 500 ml) in the urine bag attached to the bed number-2.

The detected urine bag and estimated urine level information is stored in database 514. The database 514 may be iteratively updated at a pre-defined time frame to keep track of estimated urine and understand when the urine bag is nearing full capacity. For example, the patient 502a has urine level (e.g. 1500 ml) in the urine bag attached to the bed number-1 at a specific time frame. After an hour the urine level is (e.g. 1550 ml) in the urine bag attached to bed number 1, accordingly the urine level may be updated hourly. In some embodiments, the patient 502b has urine level (e.g. 500 ml) in the urine bag attached to the bed number-2 at a specific time frame. After an hour the urine level remains (e.g. 500 ml) in the urine bag attached to bed number 2, accordingly the fluid level may be updated hourly.

In response to the updating, an issue associated with the urine level may be identified within the urine bag of at least one patient of the set of patients. For example, it may be observed that patient 502a has a urine level above the predefined threshold (e.g., 1200 ml) within the urine bag. Additionally, in certain embodiments, it may be noted that patient 502b has a urine level below the predefined threshold (e.g., 1200 ml) within the urine bag, indicating an issue such as dehydration or renal dysfunction. Moreover, there may be instances where patient 502b has passed away, leading to the absence of urine within the urine.

Further, an alert generation module 516 may generate an alert and further sends the generated alert to a radar module 518. The alert may provide real-time information about issues identified with the urine level in the urine bags of both the patients. The radar module 518 may ensure that relevant healthcare providers are notified of any abnormalities or critical issues requiring their attention. The alert is generated when the urine level estimated within the urine bag of each of the patient 502a and the patient 502b for a timeframe is above or below a pre-defined urine level. For example, if the patient 502a has been administered medications which may alter the urine output, has been diagnosed with kidney disease or diabetes insipidus, may have predefined thresholds adjusted to account for fluctuations in urine output associated with these conditions. The alert for the patient 502a may be generated if the urine level exceeds a pre-defined threshold, indicating a possible complication necessitating intervention. Additionally, if the patient 502b, has a diagnosis of acute renal failure or severe dehydration may have a predefined threshold to monitor for signs worsening disease or incomplete fluid resuscitation. An alert for the patient 502b may be generated if the urine level remains below a pre-defined threshold for an extended period, suggesting inadequate urine drainage and complications requiring intervention.

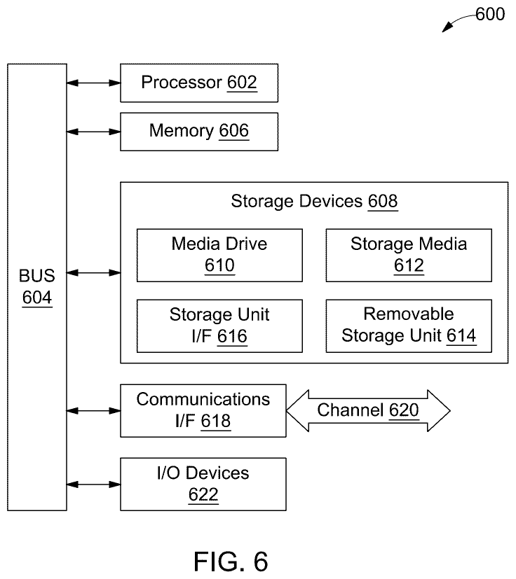

The disclosed methods and systems may be implemented on a conventional or a general-purpose computer system, such as a personal computer (PC) or server computer. Referring now to FIG. 6, an exemplary computing system 600 that may be employed to implement processing functionality for various embodiments (e.g., as a SIMD device, client device, server device, one or more processors, or the like) is illustrated. Those skilled in the relevant art will also recognize how to implement the invention using other computer systems or architectures. The computing system 600 may represent, for example, a user device such as a desktop, a laptop, a mobile phone, personal entertainment device, DVR, and so on, or any other type of special or general-purpose computing device as may be desirable or appropriate for a given application or environment. The computing system 600 may include one or more processors, such as a processor 602 that may be implemented using a general or special purpose processing engine such as, for example, a microprocessor, microcontroller, or other control logic. In this example, the processor 602 is connected to a bus 604 or other communication medium. In some embodiments, the processor 602 may be an Artificial Intelligence (AI) processor, which may be implemented as a Tensor Processing Unit (TPU), or a graphical processor unit, or a custom programmable solution Field-Programmable Gate Array (FPGA).

The computing system 600 may also include a memory 606 (main memory), for example, Random Access Memory (RAM) or other dynamic memory, for storing information and instructions to be executed by the processor 602. The memory 606 also may be used for storing temporary variables or other intermediate information during execution of instructions to be executed by the processor 602. The computing system 600 may likewise include a read only memory (“ROM”) or other static storage device coupled to bus 604 for storing static information and instructions for the processor 602.

The computing system 600 may also include storage devices 608, which may include, for example, a media drive 5610, a cloud based storage, a network storage, and a removable storage interface. The media drive 610 may include a drive or other mechanism to support fixed or removable storage media, such as a hard disk drive, a floppy disk drive, a magnetic tape drive, an SD card port, a USB port, a micro-USB, an optical disk drive, a CD or DVD drive (R or RW), or other removable or fixed media drive. A storage media 612 may include, for example, a hard disk, magnetic tape, flash drive, or other fixed or removable medium that is read by and written to by the media drive 610. As these examples illustrate, the storage media 612 may include a computer-readable storage medium having stored there in particular computer software or data.

In alternative embodiments, the storage devices 608 may include other similar instrumentalities for allowing computer programs or other instructions or data to be loaded into the computing system 600. Such instrumentalities may include, for example, a removable storage unit 614 and a storage unit interface 616, such as a program cartridge and cartridge interface, a removable memory (for example, a flash memory or other removable memory module) and memory slot, and other removable storage units and interfaces that allow software and data to be transferred from the removable storage unit 614 to the computing system 600.

The computing system 600 may also include a communications interface 618. The communications interface 618 may be used to allow software and data to be transferred between the computing system 600 and external devices. Examples of the communications interface 618 may include a network interface (such as an Ethernet or other NIC card), a communications port (such as for example, a USB port, a micro-USB port), Near field Communication (NFC), etc. Software and data transferred via the communications interface 618 are in the form of signals which may be electronic, electromagnetic, optical, or other signals capable of being received by the communications interface 618. These signals are provided to the communications interface 618 via channel 620. The channel 620 may carry signals and may be implemented using a wireless medium, wire or cable, fiber optics, or other communications medium. Some examples of the channel 620 may include a phone line, a cellular phone link, an RF link, a Bluetooth link, a network interface, a local or wide area network, and other communications channels.

The computing system 600 may further include Input/Output (I/O) devices 622. Examples may include, but are not limited to a display, keypad, microphone, audio speakers, vibrating motor, LED lights, etc. The I/O devices 622 may receive input from a user and also display an output of the computation performed by the processor 602. In this document, the terms “computer program product” and “computer-readable medium” may be used generally to refer to media such as, for example, the memory 606, the storage devices 608, the removable storage unit 614, or signal(s) on the channel 620. These and other forms of computer-readable media may be involved in providing one or more sequences of one or more instructions to the processor 602 for execution. Such instructions, generally referred to as “computer program code” (which may be grouped in the form of computer programs or other groupings), when executed, enable the computing system 600 to perform features or functions of embodiments of the present invention.

In an embodiment where the elements are implemented using software, the software may be stored in a computer-readable medium and loaded into the computing system 600 using, for example, the removable storage unit 614, the media drive 610 or the communications interface 618. The control logic (in this example, software instructions or computer program code), when executed by the processor 602, causes the processor 602 to perform the functions of the invention as described herein.

Thus, the disclosed method and system try to overcome the technical problem of detecting the fluid level in the fluid vessel connected to the patient. In contrast to sensor-based bags, which necessitate integration and incur higher initial setup or replacement costs, the disclosed method and system provides contactless and eliminates the need for hospitals to switch the types of bags they acquire. The method and system provide for continuous real-time monitoring of fluid levels in patient vessels, enabling healthcare providers to identify fluctuations or issues. Further, the method and system generate the alert and notification that may be customized based on individual patient characteristics and medical conditions, ensuring that healthcare providers receive targeted information to guide clinical decision-making. Moreover, by facilitating early detection of issues such as fluid overload or dehydration, the method and system may contribute to enhanced patient safety and reduce the likelihood of adverse events or complications.

In light of the above-mentioned advantages and the technical advancements provided by the disclosed method and system, the claimed steps as discussed above are not routine, conventional, or well understood in the art, as the claimed steps enable the following solutions to the existing problems in conventional technologies. Further, the claimed steps clearly bring an improvement in the functioning of the device itself as the claimed steps provide a technical solution to a technical problem.

It will be appreciated that, for clarity purposes, the above description has described embodiments of the invention with reference to different functional units and processors. However, it will be apparent that any suitable distribution of functionality between different functional units, processors or domains may be used without detracting from the invention. For example, functionality illustrated to be performed by separate processors or controllers may be performed by the same processor or controller. Hence, references to specific functional units are only to be seen as references to suitable means for providing the described functionality, rather than indicative of a strict logical or physical structure or organization.

Although the present invention has been described in connection with some embodiments, it is not intended to be limited to the specific form set forth herein. Rather, the scope of the present invention is limited only by the claims. Additionally, although a feature may appear to be described in connection with particular embodiments, one skilled in the art would recognize that various features of the described embodiments may be combined in accordance with the invention.

Furthermore, one or more computer-readable storage media may be utilized in implementing embodiments consistent with the present disclosure. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., be non-transitory. Examples include random access memory (RAM), read-only memory (ROM), volatile memory, nonvolatile memory, hard drives, CD ROMs, DVDs, flash drives, disks, and any other known physical storage media.

It is intended that the disclosure and examples be considered as exemplary only.

What is claimed is:

1. A method for detecting a fluid level in a fluid vessel connected to a patient, the method comprising:
receiving in real-time, by a detecting device and from at least one camera, a multimedia content associated with a set of patients within a room, wherein the multimedia content comprises at least one of an image or a video feed;
processing, by the detecting device, the multimedia content to detect a unique identifier associated with a bed of each patient of the set of patients;
analysing, by the detecting device via a first Artificial Intelligence (AI) model, the multimedia content to detect a fluid vessel attached to the bed of each patient using a pre-defined detection technique;
estimating, by the detecting device via the first AI model, the fluid level within the fluid vessel connected to each patient using a pre-defined estimation technique;
iteratively updating, by the detecting device via the first AI model, a database based on the fluid level estimated within the fluid vessel connected to each patient and the unique identifier, at a pre-defined time frame;
identifying in real-time, by the detecting device, an issue associated with the fluid level within the fluid vessel connected to at least one patient of the set of patients in response to the updating; and
generating in real-time, by the detecting device, an alert to notify at least one person about the issue associated with the fluid level within the fluid vessel connected to the at least one patient.

2. The method of claim 1, wherein the at least one camera is configured to automatically adjust to a specific position.

3. The method of claim 1, wherein processing the multimedia content comprising:
extracting information associated with each patient from the multimedia content based on the unique identifier, wherein the information includes a bed number of each patient, and medical record number (MRN) of each patient.

4. The method of claim 3, further comprising:
mapping, by the detecting device via a second AI model, the bed number to the MRN of each patient to create a mapping dataset; and
storing, by the detecting device, the mapping dataset within the database.

5. The method of claim 1, wherein the unique identifier comprises a Quick Response (QR) code, a bar code, a Radio-Frequency Identification (RFID) tag, a unique bed number, and a simultaneous localization and mapping (SLAM).

6. The method of claim 1, wherein the pre-defined detection technique comprises a Convolution Neural Network (CNN), a Region Based Convolutional Neural Network (R-CNN), a Residual Network (ResNet), a Single Shot Multibox Detector (SSD), and a You Only Look Once (YOLO).

7. The method of claim 1, wherein the pre-defined estimation technique comprises at least one of a computer vision and a machine learning.

8. The method of claim 1, wherein generating the alert comprises:
customizing alert generation based on a disease condition of each of the at least one patient.

9. The method of claim 1, wherein the alert is generated when the fluid level estimated within the fluid vessel connected to each of the at least one patient for a timeframe is above or below a pre-defined fluid level.

10. A system for detecting a fluid level in a fluid vessel connected to a patient, the system comprising:
a processor; and
a memory coupled to the processor, wherein the memory stores processor executable instructions, which, on execution, causes the processor to:
receive in real time, via at least one camera, a multimedia content associated with a set of patients within a room, wherein multimedia content comprises at least one of an image or a video feed;
process the multimedia content to detect a unique identifier associated with a bed of each patient of the set of patients;
analysis, via a first Artificial Intelligence (AI) model, the multimedia content to detect a fluid vessel attached to the bed of each patient using a pre-defined detection technique;
estimate the fluid level within the fluid vessel connected to each patient using a pre-defined estimation technique;
iteratively update, via the AI model, a database based on the fluid level estimated within the fluid vessel connected to each patient and the unique identifier, at a pre-defined time frame;
identify in real time an issue associated with the fluid level within the fluid vessel connected to at least one patient of the set of patients in response to the updating; and
generate in real time an alert to notify at least one person about the issue associated with the fluid level within the fluid vessel connected to the at least one patient.

11. The system of claim 10, wherein the at least one camera is configured to automatically adjust a current position.

12. The system of claim 10, wherein, to process the multimedia content, the processor executable instructions further cause the processor to:
extract an information associated with each patient from the multimedia content based on the unique identifier, wherein the information includes a bed number of each patient, and medical record number (MRN) of each patient.

13. The system of claim 12, wherein the processor executable instructions further cause the processor to:
map, via a second AI model, the bed number to the MRN of each patient to create a mapping dataset; and
store, the mapping dataset within the database.

14. The system of claim 10, wherein the unique identifier comprises a Quick Response (QR) code, a bar code, a Radio-Frequency Identification (RFID) tag, a unique bed number, and a simultaneous localization and mapping (SLAM).

15. The system of claim 10, wherein the pre-defined detection technique comprises a Convolution Neural Network (CNN), a Region Based Convolutional Neural Network (R-CNN), a Residual Network (ResNet), a Single Shot Multibox Detector (SSD), and a You Only Look Once (YOLO).

16. The system of claim 10, wherein the pre-defined estimation technique comprises at least one of a computer vision and a machine learning.

17. The system of claim 10, wherein generate the alert, the processor executable instructions further cause the processor to:
customize alert generation based on a disease condition of each of the at least one patient.

18. The system of claim 10, wherein the alert is generated when the fluid level estimated within the fluid vessel connected to each of the at least one patient for a timeframe is above or below a pre-defined fluid level.

19. A non-transitory computer-readable medium storing computer-executable instructions for detecting a fluid level in a fluid vessel connected to a patient, the stored instructions, when executed by a processor, cause the processor to perform operations comprises:
receiving in real-time, via at least one camera, a multimedia content associated with a set of patients within a room, wherein the multimedia content comprises at least one of an image or a video feed;
processing the multimedia content to detect a unique identifier associated with a bed of each patient of the set of patients;
analysing, via a first Artificial Intelligence (AI) model, the multimedia content to detect a fluid vessel attached to the bed of each patient using a pre-defined detection technique;
estimating, via the first AI model, the fluid level within the fluid vessel connected to each patient using a pre-defined estimation technique;
iteratively updating, via the first AI model, a database based on the fluid level estimated within the fluid vessel connected to each patient and the unique identifier, at a pre-defined time frame;
identifying in real time an issue associated with the fluid level within the fluid vessel connected to at least one patient of the set of patients in response to the updating; and
generating in real-time an alert to notify at least one person about the issue associated with the fluid level within the fluid vessel connected to the at least one patient.
